(19) United States
(12) Patent Application Publication
(10) Pub. No.: US 2025/0261855 A1
(43) Pub. Date: Aug. 21, 2025

(54) METHOD FOR CALIBRATING AND IDENTIFYING A TONOMETER PROBE

(71) Applicant: Eye to Eye Telehealth, Inc., Cheyenne, WY (US)

(72) Inventors: David Smits, Cheyenne, WY (US); Preston Smits, Denver, CO (US)

(21) Appl. No.: 18/859,332

(22) PCT Filed: Apr. 27, 2023
(86) PCT No.: PCT/US2023/020122

§ 371 Date: Oct. 23, 2024

Related U.S. Application Data
(60) Provisional application No. 63/335,312, filed on Apr. 27, 2022.

(51) Int. Cl. A61B 3/16; A61B 3/00

(52) U.S. Cl. CPC A61B 3/16; A61B 3/0025; A61B 2560/0228

(57) ABSTRACT

A method for calibrating and identifying a tonometer probe includes the steps of: a) generating an initial magnetic impulse upon a probe of ferromagnetic material via a first electromagnetic coil using known impulse parameters, b) generating a magnetic impulse via an electromagnetic coil to reverse probe velocity, c) repeating steps a)-b) for N cycles, d) measuring a resultant-induced voltage upon a measuring electromagnetic coil from probe motion, e) comparing the resultant-induced voltage upon the measuring electromagnetic coil to a reference value, and f) rejecting the probe if the induced voltage measured by the measuring electromagnetic coil is different than the reference value after N cycles.

### CROSS-REFERENCE TO RELATED APPLICATIONS

This application is a 371 National stage application based off of PCT Application No.: PCT/US2023/020122, filed 27 Apr. 2023; which claims priority to and benefit of U.S. Provisional Application No.: 63/335,312, filed 27 Apr. 2022’ of which both applications are incorporated herein.

### FIELD OF INVENTION

The present invention relates to a method for calibrating and identifying a tonometer probe for intraocular pressure measurement, and particularly with regard to magnetic saturation of the probe and induced voltage in comparison with a known reference value.

### BACKGROUND

Measuring intraocular pressure (IOP) is important for the assessment of eye health. Glaucoma is a chronic progressive blinding disease which is monitored by measuring intraocular pressure and treated by lowering intraocular pressure.

Intraocular pressure is measured with a device referred to as a tonometer. Goldman Applanation Tonometry (GAT) remains the reference-standard tonometry method and involves measuring the force required to flatten the cornea with a prism of known surface area. However, GAT has limitations to its use, requiring instillation of anesthetic drops, bulky and expensive equipment, and patient positioning difficulty. Additional methods of tonometry have been described, such as in U.S. Pat. No. 5,299,573 which uses a non-contact method wherein compressed air is sprayed on the cornea with subsequent measurement of tissue deformation.

Another method known as rebound tonometry has been described in U.S. Pat. No. 6,093,147 wherein a metal probe is set into motion by a solenoid and impacts the eye. Subsequent changes in its motion are recorded and used to derive intraocular pressure.

Rebound tonometry utilizes a probe made of ferromagnetic material which acts as a plunger within a solenoid, housed in the tonometer. As a ferromagnetic material, the probe becomes magnetized when subjected to a magnetic field and behaves like a magnet.

The degree of magnetization is determined by the probe material's magnetic parameters including magnetic saturation, magnetic remanence, magnetic induction, magnetic permeability, and magnetic field strength, and can be termed as its “magnetization value”. The magnetization value of a ferromagnetic material can range anywhere between zero and maximum, or saturation value. Under the presence of an external magnetic field, the magnetization value tends to approach the saturation value.

A brief DC induction pulse by the induction coil generates a magnetic field and will set the probe in motion, after which the same induction coil will become the measurement coil. The acceleration and velocity, collectively referred to as “driving mechanics,” of the probe are proportional to the magnetization value of the probe. This holds true when mass of the probe and pulse duration remain constant. The magnetic probe moving within the solenoid will induce a potential difference across the solenoid coil terminals, known as induced voltage. The induced voltage generated is proportional to the magnetization value of the probe and its velocity.

As the probe collides and rebounds off the surface of the eye, the induced voltage generated is proportional to IOP. This also means that derived IOP depends on the magnetization value of the probe material. As such, measurement of induced voltage is crucial to the success of rebound tonometry, and this parameter cannot be compromised. To properly convert induced voltage to IOP in a consistent and reliable manner, the ferromagnetic probe material must be nearly or fully saturated i.e., it must approach the saturation value.

If the probe material is not fully saturated, then the driving mechanics of the probe will change from measurement to measurement, leading the algorithm used to derive an incorrect IOP.

As ferromagnetic materials lose magnetization value over time, a method is required to bring the probe material to near or full saturation. Additionally, different ferromagnetic materials possess different magnetization saturation value curves, as depicted in FIG. 1. Thus, a probe made of a different ferromagnetic material will have a different magnetization saturation value curve and subsequently may lead to an incorrect IOP result as determined by the algorithm. Furthermore, there is potential for damage to the eye if the probe's magnetization value leads to increased probe velocity and impact force on the eye.

While rebound tonometry is a clinically useful technology, it requires that the correct probe be used for measurement. Specifically, use of the wrong measurement probe from an alternate rebound tonometer device or selection of other materials for use in the tonometer could have catastrophic consequences in the form of inaccurate IOP measurement or damage to the eye leading to vision loss and/or loss of the eye. Further heightening this concern is the increasing popularity of home-use rebound tonometers, in which the patient loads their own probe and checks their own eye pressure without the assistance of a licensed provider. Human factors engineering is a discipline that considers the capabilities and limitations of humans for design of safe systems and devices. It assumes that all humans, even licensed healthcare providers are fallible. Thus, there is the need for a safety method for both calibration and subsequent identification of the correct probe before the device allows the user, either healthcare provider or patient, to take a measurement.

### SUMMARY

The present invention provides a method for calibrating and identifying a tonometer probe made wholly or partially of ferromagnetic material. The method uses a tonometer device to calibrate the probe and determine whether the probe is suitable for use within a rebound tonometer based on its magnetization saturation value and induced voltage unique to the device's input parameters. More particularly, the probe must be brought to its near or full magnetic saturation value during a calibration cycle, and subsequently identify that it is the correct magnetization saturation value based on the induced voltage unique to the device's input parameters after setting the probe into motion. This verification must occur prior to beginning measurements on a patient.

The method includes the steps of: a) generating an initial magnetic impulse upon the probe via a first electromagnetic coil using known impulse parameters, b) generating a magnetic impulse via an electromagnetic coil to reverse probe velocity, c) repeating steps a)-b) for N cycles based off a known reference number of cycles, d) measuring a resultant-induced voltage upon a measuring electromagnetic coil from the motion of the probe upon an Nth cycle, e) comparing the resultant-induced voltage to a reference value, and f) rejecting the probe if the induced voltage measured by the measuring electromagnetic coil is different than the reference value after N cycles, such as more than at least 0.1 millivolt away from the reference value after N cycles.

The comparison between the resultant-induced voltage and the reference value may be carried out either before or after generating the reverse impulse. For example, rather than the above-recited order of steps, the method may include the following order of steps: a) generating an initial magnetic impulse upon the probe via a first electromagnetic coil using known impulse parameters, b) measuring a resultant-induced voltage upon a measuring electromagnetic coil from the motion of the probe, c) comparing the resultant-induced voltage to a reference value, d) generating a magnetic impulse via an electromagnetic coil to reverse probe velocity, e) repeating steps a)-d) for N cycles until the induced voltage approaches, equals, or surpasses the reference value, and f) rejecting the probe if the induced voltage measured by the measuring electromagnetic coil is different than the reference value after N cycles, such as more than at least 0.1 millivolt away from the reference value after N cycles.

The coil that provides the initial magnetic impulse may be the same coil used to measure the resultant-induced voltage. Furthermore, a single coil system may be used to provide the impulse to reverse probe velocity using the same electromagnetic coil as the measuring electromagnetic coil and initial magnetic impulse coil. Alternatively, a separate coaxial electromagnetic coil may be used to measure the resultant-induced voltage. The separate coaxial coil may also provide the impulse to reverse probe velocity, or yet another coaxial electromagnetic coil may be used to provide the impulse to reverse probe velocity. According to certain embodiments both the coil that provides the initial magnetic impulse and a separate coil may measure the resultant-induced voltage. Furthermore, the resultant-induced voltage may be measured within multiple coaxial electromagnetic coils, such as in three or more coils. Additionally, multiple electromagnetic coils may be used to provide the impulse to reverse probe velocity.

The device may be set up to use the minimum number of cycles required to achieve probe saturation, thus ending the cycles when the probe saturation is achieved. Alternatively, the device may be set up to run a set number of cycles, which may be greater than the minimum number of cycles required to reach saturation. The number of cycles may also vary with each calibration based on probe magnetization, such that the number of cycles may vary to meet the reference value specified by the tonometer design inputs.

If, upon comparing the resultant-induced voltage measured to the reference value, it is found that the induced voltage is more than a certain number of volts away from the known reference value after N cycles, then the probe is rejected. To inform the user that the probe is rejected, the device may prevent a subsequent measurement. Additionally or alternatively, the device may present a notification to the user to inform the user when the induced voltage does not approach or equal the known reference value after N cycles. The notification may be an auditory notification or a visual notification, for example.

### BRIEF DESCRIPTION OF THE DRAWINGS

FIG. 1 is a graphical representation of the magnetization saturation curves of various materials.

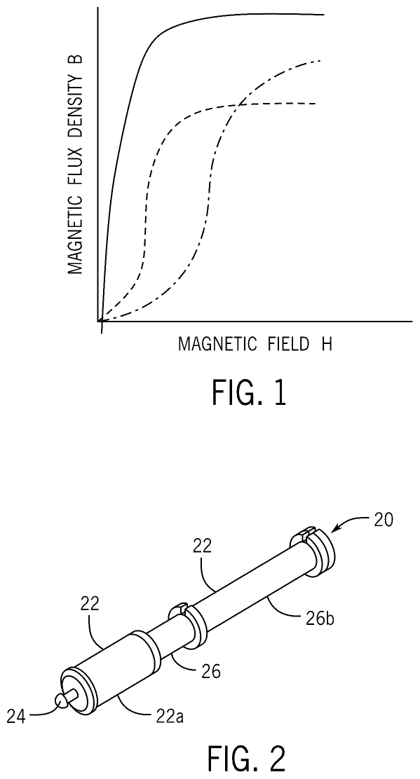

FIG. 2 is a perspective view of a coaxial two-coil system with a probe inserted.

FIG. 3 is a graphical representation of voltage applied to two separate coils.

FIGS. 4-7 are graphical representations of induced voltage over time for four different probe scenarios.

### DETAILED DESCRIPTION

Methods described herein solve the problem of probe calibration and identification for use within a rebound tonometer for measuring intraocular pressure. More specifically, by examining the probe's magnetization saturation value and induced voltage unique to the device's input parameters, the device used in conjunction with the rebound tonometer can either confirm the presence of an appropriate probe or reject an unsuitable probe.

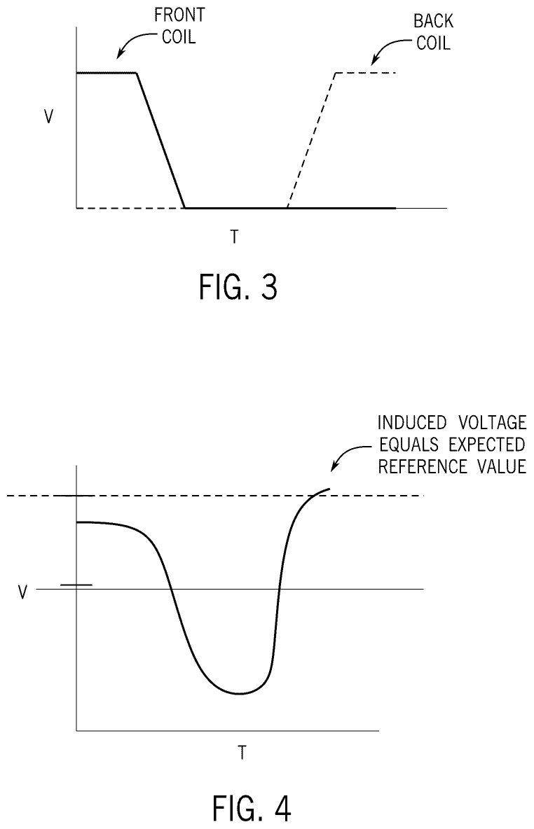

One example of a calibration and identification device 20 built into a rebound tonometer that can be used to carry out the methods is shown in FIG. 2. This device 20 has two coaxial solenoid coils 22 and a probe 24 made of partially or fully ferromagnetic material. The methods described herein can also be carried out using a similar device with a different number of solenoid coils 22, such as only one solenoid coil 22. In any case, the device 20 determines whether or not the probe 24 is suitable for use with the rebound tonometer.

The probe 24 is inserted into a shaft 26 within one or more of the solenoid coils 22, as shown in FIG. 2. The device 20 then generates an initial magnetic impulse upon the probe 24 using known impulse parameters. For instance, the device 20 can provide a controlled and pre-determined voltage and pulse duration to the outermost electromagnetic coil 22a near the point of insertion. According to other embodiments, an inner electromagnetic coil 22b, further away from the point of insertion, could provide the impulse. The impulse sets the probe 24 into motion, driving the probe 24 out of the solenoid. However, because it is undesirable to drive the probe 24 completely out of the device 20, after a very brief interval, a voltage is provided to a second coil 22 spaced apart from the first coil 22, thus reversing the probe's velocity vector and returning the probe 24 back to or near its initial starting point within the shaft 26 of the device 20. According to certain embodiments, both the initial impulse and the reverse impulse can be generated by the same coil 22. According to other embodiments, both the initial impulse and the reverse impulse can be generated by multiple coils 22.

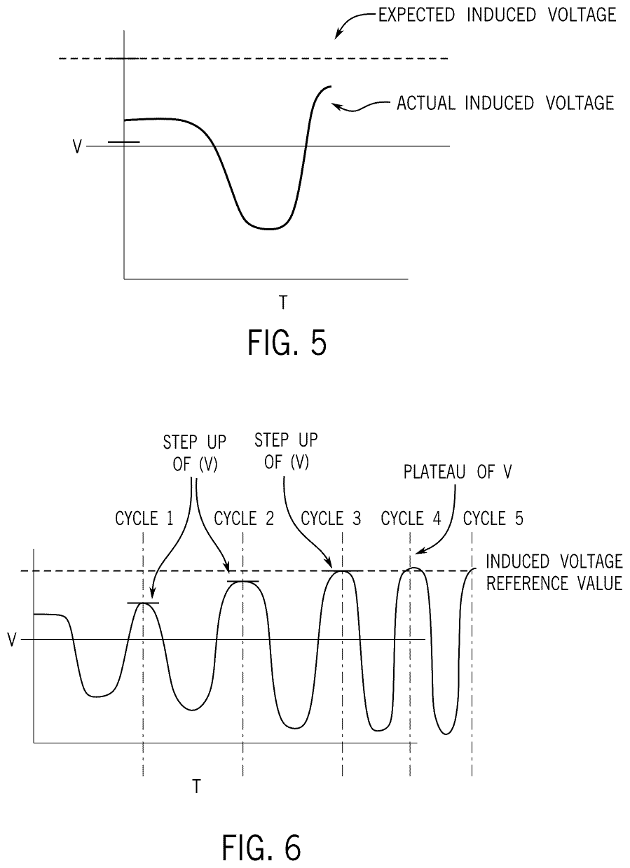

FIG. 3 is a graph depicting the voltage and pulse durations in a two-coil system during one cycle. In the brief interval where no voltage is applied, the initial impulse has been imparted upon the probe and the reverse impulse has not yet been imparted, thus the probe is moving at a specific velocity. As a ferromagnetic material, the movement of the probe within the solenoid leads to an induced voltage across the measurement coil's terminals. In this particular embodiment, the front or outermost coil is used as the measurement coil. In another embodiment, the back or inner coil could be used as the measurement coil. In yet another embodiment, the front and back coils could both be used as measurement coils. In still another embodiment, a system of multiple measurement coils, such as three or more coils, could be used.

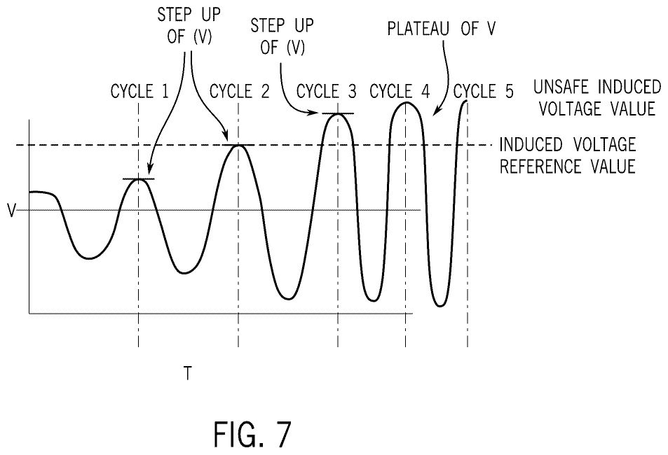

After the initial impulse and before the reverse impulse, the device “reads” the induced voltage through the one or more measuring coils. When the probe is at near or full saturation value, if the probe is an appropriate probe for use with the device, the induced voltage should equal or be within an acceptable range of values of a reference value input to the device by the manufacturer. The induced voltage reference value is specific to that device and is determined by the manufacturer from a variety of design inputs, including voltage, pulse duration, probe material, probe manufacturing process, probe magnetization value, probe weight, and required velocity and force, to name a few. As the manufacturer has designed the device to be safe and effective, comparing the induced voltage to the reference value the device is “expecting” ensures that the correct probe with the correct material and magnetization value has been loaded into the device and is calibrated. The comparison between the resultant-induced voltage and the reference value may be carried out either before or after generating the reverse impulse. If a probe is fully saturated, one cycle may be enough to ensure calibration and identification of the probe, as depicted in FIG. 4.

However, as previously described, a ferromagnetic material may lose magnetization value over time. If the probe material is not yet fully saturated, the initial impulse may lead to an induced voltage that is lower than the reference value. If calibration was completed after this single cycle and if the resultant-induced voltage upon the measuring electromagnetic coil did not approach or equal its reference value, the induced voltage and subsequent IOP determination would be incorrect during the measurement cycle taken on the patient's eye. This example is depicted in FIG. 5.

Thus, there must be a requirement of the device to check the induced voltage upon the measuring electromagnetic coil to ensure the correct probe material, and to ensure that the probe has been fully saturated. Providing additional calibration cycles, where the probe is subjected to a brief magnetic field when set into motion by the solenoid then reversed back to or near the starting position, will continually raise the probe's magnetization value to nearly or fully saturated. This will take “N” cycles, which, as described previously, is dependent on a host of parameters. During development of the probe, one can determine the number of cycles it would take to bring the probe up to saturation value if the probe is completely unmagnetized. For example, in certain embodiments, N may be between 1 and 20, or between 4 and 10, or between 5 and 8. Since the probe cannot be oversaturated, N or more cycles can be run. As the probe reaches a point of near or full saturation, if upon comparison the device reads the induced voltage upon the measuring electromagnetic coil as near or at the expected reference value, then the calibration and identification are completed and it is safe to proceed with a patient measurement using this probe. This scenario is depicted in FIG. 6 for a theoretical material and device. In the depicted example, the device utilizes more cycles than the minimum required to achieve probe saturation. In other embodiments, a device could use the minimum number of cycles required to achieve probe saturation, thus ending the cycles when the probe saturation is achieved. The number of cycles may also vary with each calibration, such that the number of cycles may be set according to one or more of the parameters or design inputs.

If, upon comparing the resultant-induced voltage measured to a reference value, it is found that the induced voltage is more than a certain number of volts away from the known reference value after N cycles, then the probe is rejected. Just as the number of cycles (N) is dependent on a host of parameters, the acceptable difference between the induced voltage and the known reference value is also dependent on a number of parameters. For example, an acceptable difference between the induced voltage and the known reference value may be up to 0.1 millivolt, or up to 1 millivolt, or up to 0.5 volt. Thus, the device may reject the probe if the induced voltage measured by the measuring electromagnetic coil is more than at least 0.1 millivolt, or more than at least 1 millivolt, or more than at least 0.5 volt away from the reference value after N cycles. To inform the user that the probe is rejected, the device may prevent a subsequent measurement. Additionally or alternatively, the device may present a notification to the user to inform the user when the induced voltage does not approach or equal the known reference value after N cycles. The notification may be an auditory notification, such as a beeping sound, or the notification may be visual, such as providing a statement or symbol on an electronic display.

In an alternative example, consider a device that does not have the probe identification process. If a user inserts a probe, wire, or material that has a greater magnetization value when fully saturated, and the device does not have a safety check by comparison to the reference value while the device impulse parameters remain the same, the velocity and induced voltage of the probe may be higher (FIG. 7). Not only will the IOP be inaccurate, but the impact force on the eye may be larger than the safety threshold, causing damage to the eye. Because there is no way to predict the present magnetization value of the probe after, for example, being left in a case for an extended amount of time, the calibration cycles to bring the probe up to saturation may be variable, and thus, without a comparison to a reference value, a device could overshoot the reference safety value upon saturation of an alternate probe material without the appropriate device feedback.

The descriptions and figures included herein depict specific implementations to teach those skilled in the art how to make and use the best option. For the purpose of teaching inventive principles, some conventional aspects have been simplified or omitted. Those skilled in the art will appreciate variations from these implementations that fall within the scope of the invention. Those skilled in the art will also appreciate that the features described above can be combined in various ways to form multiple implementations. As a result, the invention is not limited to the specific implementations described above, but only by the claims and their equivalents.

1. A method for calibrating and identifying a tonometer probe, comprising:
a) generating an initial magnetic impulse upon a probe of ferromagnetic material via a first electromagnetic coil using known impulse parameters;
b) generating a magnetic impulse via an electromagnetic coil to reverse probe velocity;
c) repeating steps a)-b) for N cycles based off a known reference number of cycles;
d) measuring a resultant-induced voltage upon a measuring electromagnetic coil from probe motion upon an Nth cycle;
e) comparing the resultant-induced voltage upon the measuring electromagnetic coil to a reference value; and
f) rejecting the probe if the induced voltage measured by the measuring electromagnetic coil is different than the reference value after N cycles.

2. The method according to claim 1, wherein the resultant-induced voltage is measured within the same coil that provided the initial magnetic impulse.

3. The method according to claim 1, wherein the resultant-induced voltage is measured within a separate coaxial electromagnetic coil.

4. The method according to claim 1, wherein the resultant-induced voltage is measured within both the same coil that provided the initial magnetic impulse and a separate coaxial electromagnetic coil.

5. The method according to claim 1, wherein the resultant-induced voltage is measured within multiple coaxial electromagnetic coils.

6. The method according to claim 1, comprising using a single coil system, and providing the impulse to reverse probe velocity using the same electromagnetic coil as the measuring electromagnetic coil and initial magnetic impulse coil.

7. The method according to claim 1, wherein the impulse to reverse probe velocity is provided by a second electromagnetic coil.

8. The method according to claim 1, wherein the impulse to reverse probe velocity is provided by multiple electromagnetic coils.

9. The method according to claim 1, wherein a number of cycles used to saturate the probe equals a minimum number of cycles required to reach saturation.

10. The method according to claim 1, wherein a number of cycles used to saturate the probe is greater than a minimum number of cycles required to reach saturation.

11. The method according to claim 1, wherein a number of cycles used to saturate the probe varies with each calibration.

12. The method according to claim 1, comprising rejecting the probe if the induced voltage measured by the measuring electromagnetic coil is more than at least 0.1 millivolt away from the reference value after N cycles.

13. The method according to claim 1, wherein when the induced voltage measured by the measuring electromagnetic coil does not approach or equal the reference value after N cycles, a subsequent measurement is not allowed.

14. The method according to claim 1, wherein when the induced voltage measured by the measuring electromagnetic coil does not approach or equal the reference value after N cycles, a notification is presented to a user.

15. The method according to claim 14, wherein the notification is an auditory notification.

16. The method according to claim 14, wherein the notification is a visual notification.

17. A method for calibrating and identifying a tonometer probe, comprising:
a) generating an initial magnetic impulse upon a probe of ferromagnetic material via a first electromagnetic coil using known impulse parameters;
b) measuring a resultant-induced voltage upon a measuring electromagnetic coil from probe motion;
c) comparing the resultant-induced voltage upon the measuring electromagnetic coil to a reference value;
d) generating a magnetic impulse via an electromagnetic coil to reverse probe velocity;
e) repeating steps a)-d) for N cycles until the induced voltage measured by the measuring electromagnetic coil approaches, equals, or surpasses the reference value; and
f) rejecting the probe if the induced voltage measured by the measuring electromagnetic coil is different than the reference value after N cycles.

18. The method according to claim 17, wherein the resultant-induced voltage is measured within the same coil that provided the initial magnetic impulse.

19. The method according to claim 17, wherein the resultant-induced voltage is measured within a separate coaxial electromagnetic coil.

20. The method according to claim 17, wherein the resultant-induced voltage is measured within both the same coil that provided the initial magnetic impulse and a separate coaxial electromagnetic coil.

21. The method according to claim 17, wherein the resultant-induced voltage is measured within multiple coaxial electromagnetic coils.

22. The method according to claim 17, comprising using a single coil system, and providing the impulse to reverse probe velocity using the same electromagnetic coil as the measuring electromagnetic coil and initial magnetic impulse coil.

23. The method according to claim 17, wherein the impulse to reverse probe velocity is provided by a second electromagnetic coil.

24. The method according to claim 17, wherein the impulse to reverse probe velocity is provided by multiple electromagnetic coils.

25. The method according to claim 17, wherein a number of cycles used to saturate the probe equals a minimum number of cycles required to reach saturation.

26. The method according to claim 17, wherein a number of cycles used to saturate the probe is greater than a minimum number of cycles required to reach saturation.

27. The method according to claim 17, wherein a number of cycles used to saturate the probe varies with each calibration.

28. The method according to claim 17, comprising rejecting the probe if the induced voltage measured by the measuring electromagnetic coil is more than at least 0.1 millivolt away from the reference value after N cycles.

29. The method according to claim 17, wherein when the induced voltage measured by the measuring electromagnetic coil does not approach or equal the reference value after N cycles, a subsequent measurement is not allowed.

30. The method according to claim 17, wherein when the induced voltage measured by the measuring electromagnetic coil does not approach or equal the reference value after N cycles, a notification is presented to a user.

31. The method according to claim 30, wherein the notification is an auditory notification.

32. The method according to claim 30, wherein the notification is a visual notification.

33. The method according to claim 17, wherein generating the magnetic impulse via the electromagnetic coil to reverse probe velocity occurs prior to comparing the resultant-induced voltage upon the measuring electromagnetic coil to the reference value.
